(19) United States
(12) Patent Application Publication
(10) Pub. No.: US 2024/0398521 A1
(43) Pub. Date: Dec. 5, 2024

(54) INTELLIGENT RESTORATION PROPOSAL

(71) Applicant: DENTSPLY SIRONA Inc., York, PA (US)

(72) Inventors: Evgenij DERZAPF, Lorsch (DE); Andre KOZA, Mannheim (DE); Daniel BIELSER, Steffisburg (CH); Simon TANAKA, Zürich (CH); Samuel MUFF, Uerikon (CH); Sandro Löschhorn, Niederglatt (CH)

(73) Assignee: DENTSPLY SIRONA Inc., York, PA (US)

(21) Appl. No.: 18/205,709

(22) Filed: Jun. 5, 2023

(51) Int. Cl. A61C 13/00; G06F 30/10

(52) U.S. Cl. CPC A61C 13/0004; G06F 30/10

(57) ABSTRACT

An intelligent restoration proposal including using an input resource to segment a 3D jaw model to obtain a segmented missing or unhealthy teeth and omitting the segmented missing or unhealthy teeth from the 3D jaw model to obtain a modified 3D jaw model. The modified 3D jaw model is used as input to a restoration proposal module to propose an output restoration. The restoration proposal module is operated as a machine learning engine. The restoration proposal is trained using a database that includes healthy teeth.

### BACKGROUND

### Technical Field

The present disclosure generally relates to restoration design and more particularly to intelligent restoration proposals using a database of healthy and/or prepared teeth.

### Description of the Related Art

In a restoration workflow, 3D images of a patient's dentition may be taken during scanning, using an intraoral camera. In a design stage of the workflow, a manual administration process may be carried out wherein tooth numbers for one or more restorations may be input and the position of one or more preparation sites may be specified on the 3D model. In the design stage, the scan may also be analyzed to generated restoration proposals using processes such as the Biogeneric method.

Following the design stage, a manufacturing stage commences wherein the generated restoration proposals may be produced by subtractive or additive manufacturing. For example, milling or grinding units may be used to produce a physical copy of the restorations. Lastly the restorations may be sintered and glazed to give the restorations their final material and esthetic properties such as hardness, strength, temperature conductivity.

### BRIEF SUMMARY

According to an embodiment of the present disclosure, a method for intelligently proposing restorations is disclosed. The method includes receiving a three-dimensional (3D) jaw model of a patient, the 3D jaw model including at least one missing or prepared tooth. An input resource may segment the 3D jaw model to obtain at least one segmented missing or prepared tooth area. The segmentation may be optional. Responsive to the 3D jaw model including at least one missing tooth, the at least one segmented missing tooth area may be removed from the 3D jaw model to obtain a modified 3D jaw model. Responsive to the 3D jaw model including at least one prepared tooth, a position of the at least one prepared tooth area may be indicated to obtain the modified 3D jaw model. Using the restoration proposal module, and the modified 3D jaw model as input to the restoration proposal module, at least one output restoration may be proposed for the at least one segmented missing or prepared tooth. The restoration proposal module may comprise, for example, two restoration proposal modules that are separately trained for predictions based on missing tooth and for predictions based on prepared tooth, respectively. The restoration proposal module may alternatively be a single restoration proposal module. The restoration proposal module may be operated as a machine learning engine. This may be significantly beneficial as the more data available for use in training a machine learning model, the better the proposals obtained. Since there may not be enough already designed restorations for use in training a model, using healthy teeth in training the model may offer the ability to obtain large numbers of training data for training, validation and testing.

In an embodiment, a training of a restoration proposal module may be done by receiving a plurality of training 3D jaw models that each include at least one healthy and/or prepared training tooth. For each training 3D jaw model of the plurality of training 3D jaw models, the following may be performed. The training 3D jaw model may be segmented to identify at least one healthy and/or prepared training segmented tooth area corresponding to the at least one healthy and/or prepared training tooth. The segmentation may be optional. Responsive to the training 3D jaw model including at least one healthy training segmented tooth, at least one healthy training segmented tooth area may be omitted from the training 3D jaw model to obtain a modified training 3D jaw model. Responsive to the training 3D jaw model including at least one prepared training tooth, a position of the at least one prepared training segmented tooth area may be indicated to obtain the modified training 3D jaw model. The modified training 3D jaw model may be provided as input to the restoration proposal module and the restoration proposal module may propose at least one corresponding training output restoration for the at least one healthy and/or prepared training segmented tooth area. A difference between the at least one healthy and/or prepared training segmented tooth and the at least one corresponding training output restoration may be computed and parameters of the restoration proposal module may be updated accordingly. The proposing may be repeated until the measured distance/difference is minimized.

According to an embodiment of the present disclosure, a non-transitory computer-readable storage medium is disclosed. The computer-readable storage medium may include instructions that when executed by a computer, cause the computer to carry out a method that includes receiving a three-dimensional (3D) jaw model of a patient, the 3D jaw model including at least one missing or prepared tooth. An input resource may segment the 3D jaw model to obtain at least one segmented missing or prepared tooth area. The segmentation may be optional. Responsive to the 3D jaw model including at least one missing tooth, the at least one segmented missing tooth area may be from the 3D jaw model to obtain a modified 3D jaw model. Responsive to the 3D jaw model including at least one prepared tooth, a position of the at least one prepared tooth area may be indicated to obtain the modified 3D jaw model. Using the restoration proposal module, and the modified 3D jaw model as input to the restoration proposal module, at least one output restoration may be proposed for the at least one segmented missing or prepared tooth.

According to an embodiment of the present disclosure, a computing system is disclosed. The computing system may include a processor and memory storing instructions that, when executed by the processor, configure the system to receive a three-dimensional (3D) jaw model of a patient, the 3D jaw model including at least one missing or prepared tooth; segment, by an input resource, the 3D jaw model to obtain at least one segmented missing or prepared tooth area; responsive to the 3D jaw model including at least one missing tooth, omit, the at least one segmented missing tooth area from the 3D jaw model to obtain a modified 3D jaw model; responsive to the 3D jaw model including at least one prepared tooth, indicate a position of the at least one prepared tooth area to obtain the modified 3D jaw model; and propose, using the restoration proposal module, and the modified 3D jaw model as input to the restoration proposal module, at least one output restoration for the at least one segmented missing or prepared tooth.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 depicts a block diagram of a network of data processing systems in which illustrative embodiments may be implemented.

FIG. 2 depicts a block diagram of a data processing system in which illustrative embodiments may be implemented.

FIG. 3 depicts a block diagram of an example configuration for intelligent proposal of restorations and dental/workflow parameters for machining in accordance with one or more illustrative embodiments.

FIG. 4 depicts a block diagram illustrating a training system in which illustrative embodiments may be implemented.

FIG. 5 depicts a sketch of a training method for proposal restorations in accordance with one or more illustrative embodiments.

FIG. 6 depicts a block diagram of an example training architecture for machine-learning based recommendation engine in accordance with one or more illustrative embodiments.

FIG. 7 depicts a routine in accordance with one or more illustrative embodiments.

### DETAILED DESCRIPTION

The illustrative embodiments recognize that the manual placement of restorations or selection of restoration types occurs at two points in a restoration workflow, once at the start of a design stage in the workflow by the definition of tooth number and tooth indication using on a tooth diagram or tooth scheme and then after the generation of a three-dimensional (3D) model using the 3D jaw's scanned regions, the restoration's location is entered or selected (usually by specifying the preparation margin). The illustrative embodiments recognize that this is not only time-consuming but also error-prone, especially for new dental professionals.

The illustrative embodiments further recognize that in conventional software, a number of already generated restorations may be accumulated and used as a basis for creating new restorations, based on for example, matching algorithms. However, this relies on the presence of existing restorations such as crowns, inlays etc in a database and as such not having such existing restorations as a starting point to generate new restorations may be significantly difficult to achieve. Furthermore, artificial designed restorations may vary widely from real morphologies.

The illustrative embodiments employ the use of databases containing healthy and/or prepared teeth such as prepared tooth stumps or other preparation types to train a restoration proposal module to propose new restorations. Advantageously, a need for input from already designed restorations may be alleviated or even eliminated. Even further, using a database of continuously changing three-dimensional (3D) scans of teeth that are more readily available and in larger numbers may make the proposal of restorations for replacing missing or removed teeth more accurate and expeditious for dental practitioners.

The illustrative embodiments disclose a method that comprises receiving a 3D jaw model of a patient, the 3D jaw model including at least one missing or prepared tooth; segmenting, by an input resource, the 3D jaw model to obtain at least one segmented missing or prepared tooth; omitting, for the 3D jaw model that includes an identified segmented missing tooth, the at least one segmented missing tooth from the 3D jaw model to obtain a modified 3D jaw model. For the 3D jaw model that includes an identified segmented prepared tooth the 3D jaw model may be the same as the modified 3D jaw model with a position of the prepared tooth indicated. Using the restoration proposal module, and the modified 3D jaw model as input to the restoration proposal module, at least one output restoration may be proposed to fill the omitted at least one segmented missing or prepared tooth, wherein the restoration proposal module is operated as a machine learning engine.

The illustrative embodiments train the restoration proposal module by receiving a plurality of training 3D jaw models that include healthy and/or prepared teeth. For each training 3D jaw model of the plurality of training 3D jaw models, the training 3D jaw model is segmented to identify at least one healthy training segmented tooth or at least one prepared training segmented tooth from the remaining teeth; which is omitted or a position thereof indicated respectively to obtain a modified training 3D jaw model. In a scenario where segmentation is not performed, the training 3D jaw model may be provided as is to the machine learning model for predictions and a relatively large number of samples may be used for training. The modified training 3D jaw model is provided as input to the restoration proposal module; the restoration proposal module is used to propose at least one corresponding training output restoration for the at least one healthy training segmented teeth that is omitted; a difference between the at least one healthy and/or or prepared training segmented teeth and corresponding training output restoration(s) is measured, parameters of the restoration proposal module are updated and the proposing may be repeated until the measured distance is minimized. Other technical features may be readily apparent to one skilled in the art from the following figures, descriptions, and claims.

The illustrative embodiments are described with respect to certain types of data, functions, algorithms, equations, model configurations, locations of embodiments, additional data, devices, data processing systems, environments, components, and applications only as examples. Any specific manifestations of these and other similar artifacts are not intended to be limiting to the invention. Any suitable manifestation of these and other similar artifacts can be selected within the scope of the illustrative embodiments.

Furthermore, the illustrative embodiments may be implemented with respect to any type of data, data source, or access to a data source over a data network. Any type of data storage device may provide the data to an embodiment of the invention, either locally at a data processing system or over a data network, within the scope of the invention. Where an embodiment is described using a mobile device, any type of data storage device suitable for use with the mobile device may provide the data to such embodiment, either locally at the mobile device or over a data network, within the scope of the illustrative embodiments.

The illustrative embodiments are described using specific code, designs, architectures, protocols, layouts, schematics, and tools only as examples and are not limiting to the illustrative embodiments. Furthermore, the illustrative embodiments are described in some instances using particular software, tools, and data processing environments only as an example for the clarity of the description. The illustrative embodiments may be used in conjunction with other comparable or similarly purposed structures, systems, applications, or architectures. For example, other comparable devices, structures, systems, applications, or architectures therefore, may be used in conjunction with such embodiment of the invention within the scope of the invention. An illustrative embodiment may be implemented in hardware, software, or a combination thereof.

The examples in this disclosure are used only for the clarity of the description and are not limiting to the illustrative embodiments. Additional data, operations, actions, tasks, activities, and manipulations will be conceivable from this disclosure and the same are contemplated within the scope of the illustrative embodiments.

Any advantages listed herein are only examples and are not intended to be limiting to the illustrative embodiments. Additional or different advantages may be realized by specific illustrative embodiments. Furthermore, a particular illustrative embodiment may have some, all, or none of the advantages listed above.

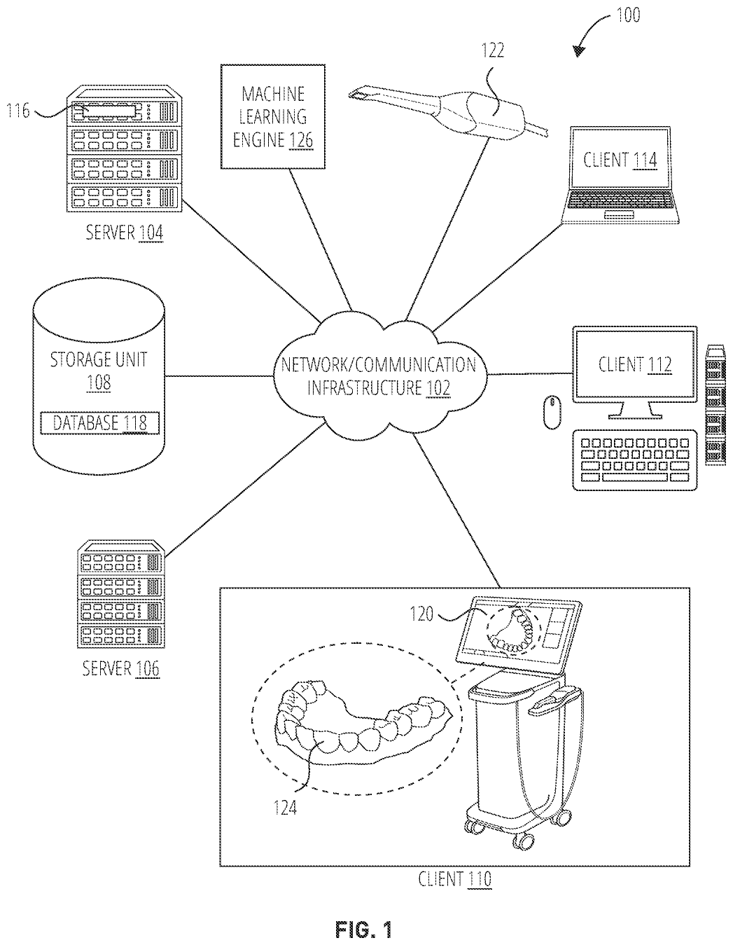

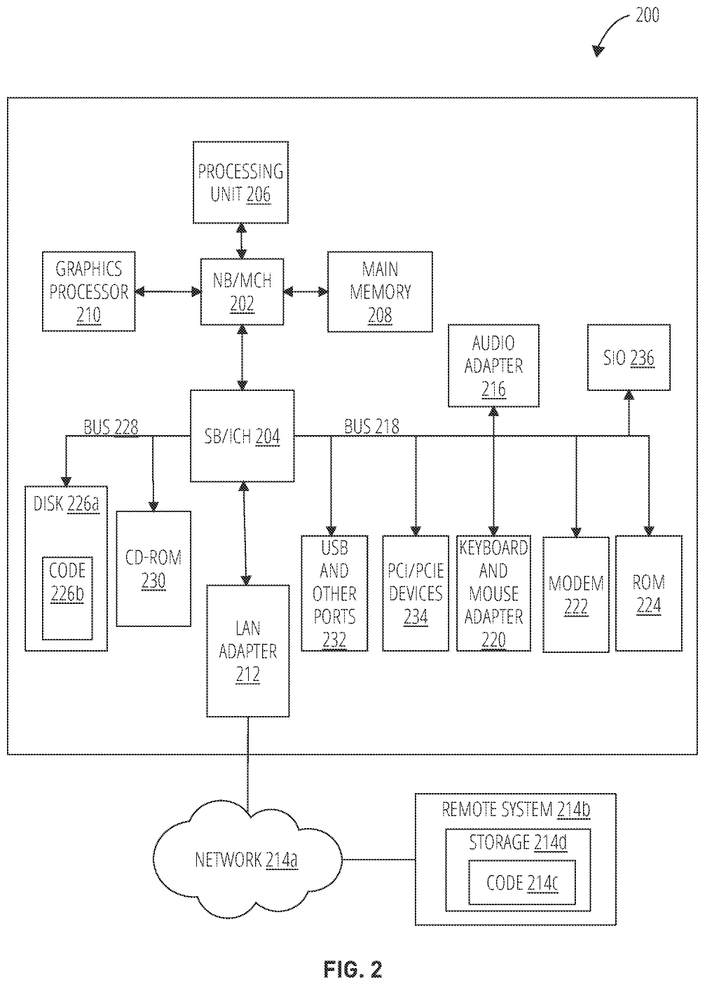

With reference to the figures and in particular with reference to FIG. 1 and FIG. 2, these figures are example diagrams of data processing environments in which illustrative embodiments may be implemented. FIG. 1 and FIG. 2 are only examples and are not intended to assert or imply any limitation with regard to the environments in which different embodiments may be implemented. A particular implementation may make many modifications to the depicted environments based on the following description.

FIG. 1 depicts a block diagram of a network of data processing systems in which illustrative embodiments may be implemented. Data processing environment is a network of computers in which the illustrative embodiments may be implemented. Data processing environment 100 includes network/communication infrastructure 102. Network/communication infrastructure 102 is the medium used to provide communications links between various devices, databases and computers connected together within data processing environment 100. Network/communication infrastructure 102 may include connections, such as wire, wireless communication links, or fiber optic cables.

Clients or servers are only example roles of certain data processing systems connected to network/communication infrastructure 102 and are not intended to exclude other configurations or roles for these data processing systems. Server 104 and server 106 couple to network/communication infrastructure 102 along with storage unit 108. Software applications may execute on any computer in data processing environment 100. Client 110, client 112, client 114 are also coupled to network/communication infrastructure 102. Client 110 may be a dental acquisition unit with a display. A data processing system, such as server 104 or server 106, or clients (client 110, client 112, client 114) may include data and may have software applications or software tools executing thereon.

Only as an example, and without implying any limitation to such architecture, FIG. 1 depicts certain components that are usable in an example implementation of an embodiment. For example, servers and clients are only examples and do not imply a limitation to a client-server architecture. As another example, an embodiment can be distributed across several data processing systems and a data network as shown, whereas another embodiment can be implemented on a single data processing system within the scope of the illustrative embodiments. Data processing systems (server 104, server 106, client 110, client 112, client 114) also represent example nodes in a cluster, partitions, and other configurations suitable for implementing an embodiment.

Dental scanner 122 includes one or more sensors that may measure or scan tooth geometry and/or color by obtaining a plurality of images through projections and combining the projections to obtain a three-dimensional (3D) image. In an example, the dental scanner 122 captures data points as often as several thousand times each second, automatically registering the sizes and shapes of each tooth. It continuously sends this data to the connected computer's software, which builds it into a 3D impression of the patient's oral cavity.

A most widely used digital format is the STL (Standard Tessellation Language) format. This format describes a succession of triangulated surfaces where each triangle is defined by three points and a normal surface. STL files may describe only the surface geometry of a three-dimensional object without any representation of color, texture or other CAD model attributes. However, other file formats have been developed to record color, transparency, or texture of dental tissues (such as Polygon File Format, PLY files) and may also be used. Irrespective of the type of imaging technology employed, scanners or cameras project light that is then recorded as individual images and compiled by the software after recognition of POI (points of interest). For example, two coordinates (x and y) of each point are evaluated on the image, and the third coordinate (z) is then calculated depending on a distance from the scanner.

Client application 120 or any other server application 116 implements an embodiment described herein. Client application 120 and/or server application 116 can use data from dental scanner 122 to generate 3D jaw models of one or more teeth 124 that may be used for training a restoration proposal module. They may also derive input data from a database 118.

Client application 120 can also execute in any of data processing systems (server 104 or server 106, client 110, client 112, client 114), such as client server application 116 in server 104 and need not execute in the same system as client 110.

Machine learning engine 126 may perform one or more intelligent restoration proposals and/or training methods as discussed herein. Machine learning engine 126 may be a part of, or separate from server 104 or clients 110, 112 and 114.

Server 104, server 106, storage unit 108, client 110, client 112, client 114, may couple to network/communication infrastructure 102 using wired connections, wireless communication protocols, or other suitable data connectivity. Client 110, client 112 and client 114 may be, for example, personal computers or network computers.

In the depicted example, server 104 may provide data, such as boot files, operating system images, and applications to client 110, client 112, and client 114. Client 110, client 112 and client 114 may be clients to server 104 in this example. Client 110, client 112 and client 114 or some combination thereof, may include their own data, boot files, operating system images, and applications. Data processing environment 100 may include additional servers, clients, and other devices that are not shown. Server 104 includes the server application 116 that may be configured to implement one or more of the functions described herein for displaying restoration proposals in accordance with one or more embodiments.

Server 106 may include a search engine configured to search stored files such as images, 3D models of patients and preferences for a dental practice in response to a request from an operator as described herein with respect to various embodiments.

In the depicted example, data processing environment 100 may be the Internet. Network/communication infrastructure 102 may represent a collection of networks and gateways that use the Transmission Control Protocol/Internet Protocol (TCP/IP) and other protocols to communicate with one another. At the heart of the Internet is a backbone of data communication links between major nodes or host computers, including thousands of dental practices, commercial, governmental, educational, and other computer systems that route data and messages. Of course, data processing environment 100 also may be implemented as a number of different types of networks, such as for example, an intranet, a local area network (LAN), or a wide area network (WAN). FIG. 1 is intended as an example, and not as an architectural limitation for the different illustrative embodiments.

Among other uses, data processing environment 100 may be used for implementing a client-server environment in which the illustrative embodiments may be implemented. A client-server environment enables software applications and data to be distributed across a network such that an application functions by using the interactivity between a client data processing system and a server data processing system. Data processing environment 100 may also employ a service-oriented architecture where interoperable software components distributed across a network may be packaged together as coherent business applications. Data processing environment 100 may also take the form of a cloud, and employ a cloud computing model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service.

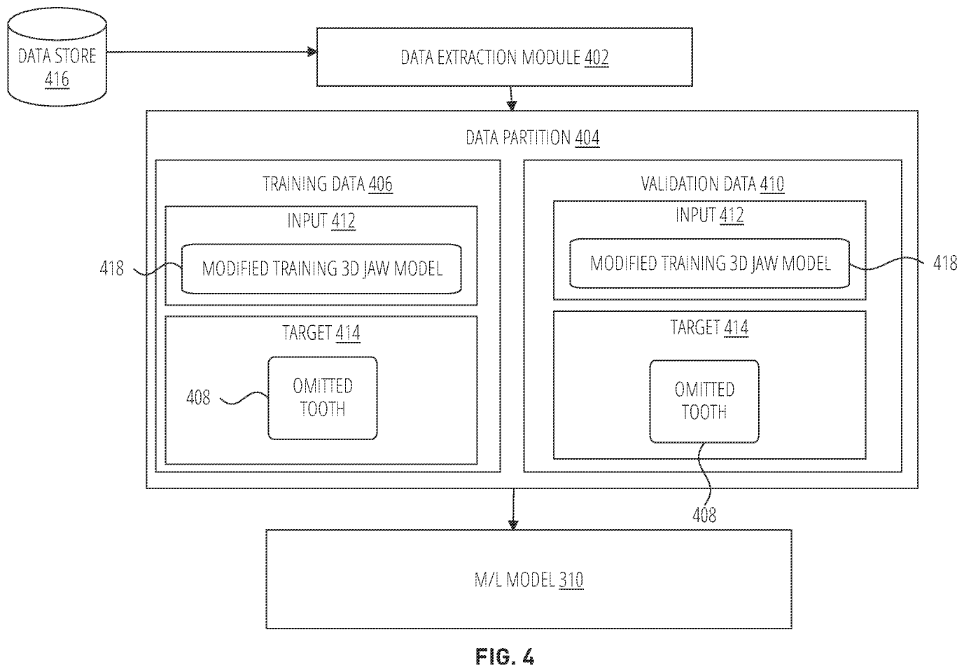

With reference to FIG. 2, this figure depicts a block diagram of a data processing system in which illustrative embodiments may be implemented. Data processing system 200 is an example of a computer, such client 110, client 112, client 114 or server 104, server 106, in FIG. 4, or another type of device in which computer usable program code or instructions implementing the processes may be located for the illustrative embodiments.

Data processing system 200 is described as a computer only as an example, without being limited thereto. Implementations in the form of other devices, may modify data processing system 200, such as by adding a touch interface, and even eliminate certain depicted components from data processing system 200 without departing from the general description of the operations and functions of data processing system 200 described herein.

In the depicted example, data processing system 200 employs a hub architecture including North Bridge and memory controller hub (NB/MCH) 202 and South Bridge and input/output (I/O) controller hub (SB/ICH) 204. Processing unit 206, main memory 208, and graphics processor 210 are coupled to North Bridge and memory controller hub (NB/MCH) 202. Processing unit 206 may include one or more processors and may be implemented using one or more heterogeneous processor systems. Processing unit 206 may be a multi-core processor. Graphics processor 210 may be coupled to North Bridge and memory controller hub (NB/MCH) 202 through an accelerated graphics port (AGP) in certain implementations.

In the depicted example, local area network (LAN) adapter 212 is coupled to South Bridge and input/output (I/O) controller hub (SB/ICH) 204. Audio adapter 216, keyboard and mouse adapter 220, modem 222, read only memory (ROM) 224, universal serial bus (USB) and other ports 232, and PCI/PCIe devices 234 are coupled to South Bridge and input/output (I/O) controller hub (SB/ICH) 204 through bus 218. Hard disk drive (HDD) or solid-state drive (SSD) 226a and CD-ROM 230 are coupled to South Bridge and input/output (I/O) controller hub (SB/ICH) 204 through bus 228. PCI/PCIe devices 234 may include, for example, Ethernet adapters, add-in cards, and PC cards for notebook computers. PCI uses a card bus controller, while PCIe does not. Read only memory (ROM) 224 may be, for example, a flash binary input/output system (BIOS). Hard disk drive (HDD) or solid-state drive (SSD) 226a and CD-ROM 230 may use, for example, an integrated drive electronics (IDE), serial advanced technology attachment (SATA) interface, or variants such as external-SATA (eSATA) and micro-SATA (mSATA). A super I/O (SIO) device 236 may be coupled to South Bridge and input/output (I/O) controller hub (SB/ICH) 204 through bus 218.

Memories, such as main memory 208, read only memory (ROM) 224, or flash memory (not shown), are some examples of computer usable storage devices. Hard disk drive (HDD) or solid-state drive (SSD) 226a, CD-ROM 230, and other similarly usable devices are some examples of computer usable storage devices including a computer usable storage medium.

An operating system runs on processing unit 206. The operating system coordinates and provides control of various components within data processing system 200 in FIG. 2. The operating system may be a commercially available operating system for any type of computing platform, including but not limited to server systems, personal computers, and mobile devices. An object oriented or other type of programming system may operate in conjunction with the operating system and provide calls to the operating system from programs or applications executing on data processing system 200.

Instructions for the operating system, the object-oriented programming system, and applications or programs, such as server application 116 and client application 120 in FIG. 1, are located on storage devices, such as in the form of codes 226b on Hard disk drive (HDD) or solid-state drive (SSD) 226a, and may be loaded into at least one of one or more memories, such as main memory 208, for execution by processing unit 206. The processes of the illustrative embodiments may be performed by processing unit 206 using computer implemented instructions, which may be located in a memory, such as, for example, main memory 208, read only memory (ROM) 224, or in one or more peripheral devices.

Furthermore, in one case, code 226b may be downloaded over network 214a from remote system 214b, where similar code 214c is stored on a storage device 214d in another case, code 226b may be downloaded over network 214a to remote system 214b, where downloaded code 214c is stored on a storage device 214d.

The hardware in FIG. 1 and FIG. 2 may vary depending on the implementation. Other internal hardware or peripheral devices, such as flash memory, equivalent non-volatile memory, or optical disk drives and the like, may be used in addition to or in place of the hardware depicted in FIG. 1 and FIG. 2. In addition, the processes of the illustrative embodiments may be applied to a multiprocessor data processing system.

In some illustrative examples, data processing system 200 may be a personal digital assistant (PDA), which is generally configured with flash memory to provide non-volatile memory for storing operating system files and/or user-generated data. A bus system may comprise one or more buses, such as a system bus, an I/O bus, and a PCI bus. Of course, the bus system may be implemented using any type of communications fabric or architecture that provides for a transfer of data between different components or devices attached to the fabric or architecture.

A communications unit may include one or more devices used to transmit and receive data, such as a modem or a network adapter. A memory may be, for example, main memory 208 or a cache, such as the cache found in North Bridge and memory controller hub (NB/MCH) 202. A processing unit may include one or more processors or CPUs.

The depicted examples in FIG. 1 and FIG. 2 and above-described examples are not meant to imply architectural limitations. For example, data processing system 200 also may be a tablet computer, laptop computer, or telephone device in addition to taking the form of a mobile or wearable device.

Where a computer or data processing system is described as a virtual machine, a virtual device, or a virtual component, the virtual machine, virtual device, or the virtual component operates in the manner of data processing system 200 using virtualized manifestation of some or all components depicted in data processing system 200. For example, in a virtual machine, virtual device, or virtual component, processing unit 206 is manifested as a virtualized instance of all or some number of hardware processing units 206 available in a host data processing system, main memory 208 is manifested as a virtualized instance of all or some portion of main memory 208 that may be available in the host data processing system, and Hard disk drive (HDD) or solid-state drive (SSD) 226a is manifested as a virtualized instance of all or some portion of Hard disk drive (HDD) or solid-state drive (SSD) 226a that may be available in the host data processing system. The host data processing system in such cases is represented by data processing system 200.

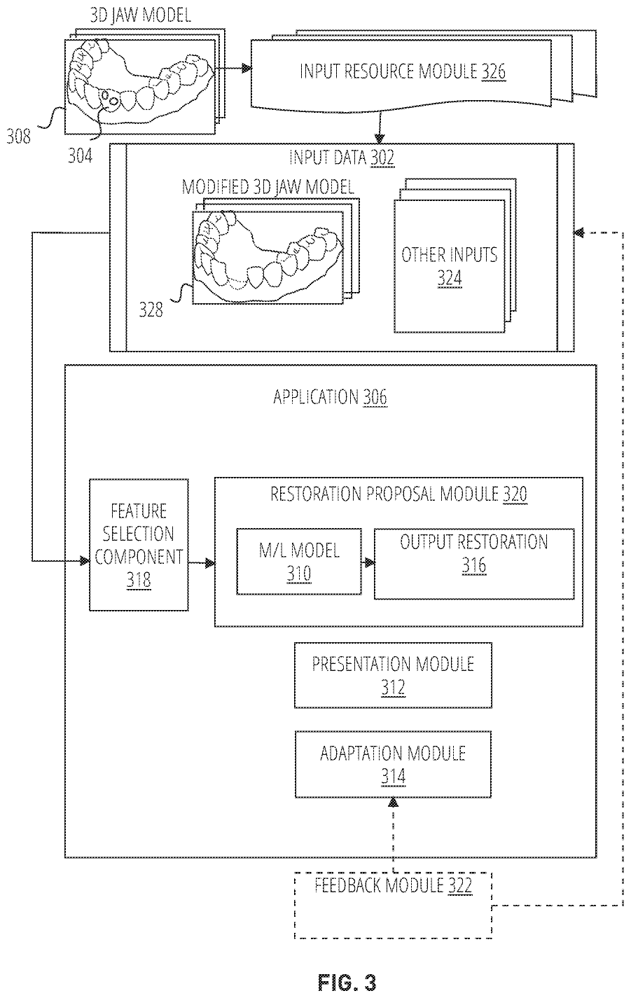

With reference to FIG. 3, this figure depicts a block diagram of an example configuration for intelligent proposal of restorations in accordance with an illustrative embodiment. Application 306 is an example of any of server applications server application 116 or client application 120, depending on the particular implementation.

In one aspect, application 306 may receive a three-dimensional (3D) scan (3D jaw model) of a dental jaw in need of a restoration and may propose a restoration using the restoration proposal module 320 which may be a trained machine learning model that may be trained as shown in FIG. 4. Prior to the application 306 receiving the 3D jaw model 308, the 3D jaw model 308 may be preprocessed by an input resource module 326 into a modified 3D jaw model 328. The input resource module 326 may obtain the 3D jaw model 308 and identify a missing or prepared tooth 304. In the case of a missing tooth, an area of the missing tooth may be segmented and removed from the 3D jaw model 308 and the 3D jaw model 308 along with an indication of the location may be provided as a modified 3D jaw model 328 to the application 306. Alternatively, or in addition, a prepared tooth such as a prepared tooth stump may be identified from the segmentation and a location thereof along with the jaw model may be provided as input to the application 306. Of course, embodiments may be realized in view of the descriptions herein, wherein segmentation may be bypassed. Further, an AI model may be trained for predictions based on jaw models including missing teeth and a separate AI model may alternatively be trained for predictions based on jaw models including prepared tooth stumps. There may be several approaches for the segmentation. In one example, segmentation may be performed by determining a jaw line along the jaw model and finding the tooth regions starting with sample points over the tooth surface by the “max-flow min cut” optimization algorithm. Alternatively, a separate AI model may be configured to directly learn which region on a jaw scan corresponds to which tooth.

Moreover, the application 306 may additionally take one or more other inputs 324 including but not limited to a) an estimation of a jaw line of the 3D jaw model obtained, for example, by computing a midline along a contour of the 3D jaw model; b) extracted feature information from the 3D jaw model, the extracted features including, for example, cusps, fissures, morphology of the teeth, etc.; c) a categorization of one or more teeth of the 3D jaw model as healthy or previously unhealthy but currently prepared tooth, using for example, a trained AI which is trained based on height fields (rasterized height maps) or trained on real 3d surfaces; d) computed orientation of the 3D jaw model or computed tooth axis; e) computed tooth number for one or more teeth of the 3D jaw model; f) computed one or more colors of a tooth of the 3D jaw model, g) an identification of the tooth to be treated. The computations of the other inputs 324 may be performed by the input resource module 326.

The input data 302 may be used as input for a restoration proposal module 320 which may then propose at least one output restoration 316 to replace the missing or prepared tooth 304. The restoration proposal module 320, may comprise a trained deep neural network/m/l model 310 (machine learning model).

The input resource module 326 may automatically detect a drilled cavity in an image to identify a missing tooth, or a preparation site, by for example, image analysis and may automatically segment portions of the teeth or oral cavity to limit a subsequent analysis, by the deep neural network described herein, to neighboring teeth and/or portions of the preparation site such as a tooth stump. The input resource module 326 may alternatively automatically detect a prepared tooth by image analysis. Thus, the input data may be selected to be representative of an absent tooth obtained from a segmentation process, i.e., the input data includes physical characteristics of one or more portions of an oral cavity including, for example, the empty area of the jaw model and neighboring teeth, and at least one output restoration characteristic value may be proposed based on a segmented portion.

In an embodiment, the application 306 may include a feature selection component 318 that may be configured to generate relevant features for a proposal based on data from all the different available inputs (e.g., 3D jaw model 308, other inputs 324 such as cusps, fissures, morphology, tooth color, etc). The feature selection component 318 may be a component outside the deep neural network and may receive a request from application 306 which may include at least an identification of a recommendation/proposal type needed (e.g., an indication that the 3D jaw model includes at least one prepared tooth, rather than a missing tooth. In a case where the missing or prepared tooth 304 is not automatically computed, a request to propose a restoration for a tooth number Y may be given, or in another example, a request to propose a restoration for any one or more unhealthy or missing teeth that may be present in a scanned jaw using at least caries detection data may be given. In the embodiment, feature selection component 318 may use a defined algorithm of prioritization or dependencies to generate the features as input for the restoration proposal module 320. In another embodiment, feature selection component 318 may be absent and the input data 302 may be provided directly to the restoration proposal module 320 to propose the output restoration 316. The output restoration 316 may have features that match features of the surrounding tooth in the jaw model and/or features of a typical tooth in the position of the removed tooth and may be devoid of the unhealthy features detected in the missing or unhealthy tooth 304.

The output restoration 316 may also comprise a proposal of, for example, one or more manufacturing process and/or restoration specification for use in controlling a machine to manufacture the output restoration 316. The machine may be an additive manufacturing machine such as a 3D Printer or a subtractive manufacturing machine such as a milling or grinding unit. The machine may use information such as the dimensions of the output restoration 316 or the other output data for manufacturing the output restoration 316.

The restoration proposal module 320 can be based, for example, on an artificial machine learning neural network such as a convolutional neural network (CNN) or Pointnet though these are not meant to be limiting. A training of the m/l model 310 or restoration proposal module 320 according to an illustrative embodiment is discussed hereinafter.

In an illustrative embodiment, presentation module 312 of application 306 displays proposals obtained from the restoration proposal module 320. The presentation module 312 may display, for example, one or a plurality of output restorations 316 for one or a plurality of missing or prepared teeth. An adaptation module 314 may be configured to receive input from the practitioner to adapt the output restorations 316 if necessary. For example, indicating a correctness of the output may cause a recalculation of proposed output restorations 316 for presentation by the presentation module 312.

As new patient scans and other input data are added to the database 118, retraining of the restoration proposal module 320 may be manually or automatically initiated. Scans of real patients may be collected during use of the system discussed herein and added to an existing database. Feedback module 322 optionally collects user feedback 324 indicative of an accuracy of the output restorations 316 for retraining the restoration proposal module 320 or for altering a processing of the input data. Advantageously, a large number of healthy jaw models may be used to train the proposal model by providing a local coordinate system and some tooth features around a proposal area.

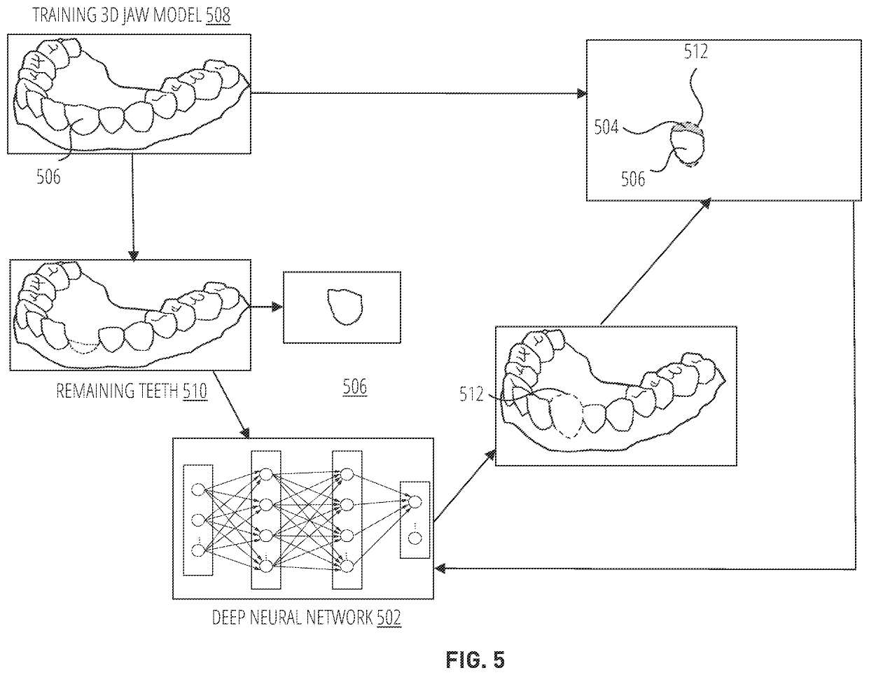

FIG. 4 shows a block diagram illustrating a training of a machine learning model. The training may be performed by the machine learning engine 126 which may extract, by data extraction module 402, a plurality of training 3D jaw models from the dataset of data store 416 for use in training the m/l model 310. The plurality of training 3D jaw models may each be modified into a plurality of modified training 3D jaw model 418. More specifically, for each training 3D jaw model of the plurality of training 3D jaw models, the training 3D jaw model may be segmented to identify a healthy tooth 506 (a healthy training segmented tooth, see FIG. 5) from the remaining teeth 510. In training a machine learning model for predictions based on missing teeth, the healthy training segmented tooth may be omitted (as the healthy tooth 506/omitted tooth 408) from the training 3D jaw model to obtain a modified training 3D jaw model 418 in FIG. 4 or remaining teeth 510 in FIG. 5. The modified training 3D jaw model 418 may be provided as input to the m/l model 310 which may be a deep neural network 502 and which may propose, a corresponding training output restoration 512. In training a machine learning model for predictions based on prepared tooth stumps, a practitioner may obtain a physical model of the healthy tooth with or without surrounding teeth. The actual healthy teeth in a patient may alternatively be used. A pre-operation scan of the healthy tooth may be obtained. The practitioner may then drill out portions of the healthy tooth to obtain a tooth preparation, e.g. a tooth stump. A post operation scan of the prepared tooth with or without surrounding teeth may then be obtained. The virtual representation of the prepared tooth stump may be used for the training by providing the post operation scan or a modified version thereof as the modified training 3D jaw model 418 which is in turn provided as input to the m/l model 310 to propose a corresponding training output restoration 512. Due to the tooth preparation having some known characteristics, such as a width and position of the base of the tooth neck, additional features may be available for use by the m/l model 310 in making more structurally accurate predictions. A difference 504 between the omitted tooth 408 or healthy tooth 506 (which is in the case of the prepared tooth obtained from the pre-operative scan/3D image) and the training output restoration 512 may be measured for use in updating parameters of the m/l model 310. In one illustrative example, a number of samples on the surface of one of the geometries may be determined and searches for corresponding positions on the other surface by projection or nearest neighboring search may be performed. Moreover, with such corresponding pairs a metric such as averaging or maximizing of the distances between the corresponding samples may be defined. Of course, other methods may be used to measure the difference 504 in light of the descriptions herein. The proposal may be repeated with the updated parameters until the measured distance is minimized. The difference 504 may be based on relative distances of the omitted tooth 408 or healthy tooth 506 from the training output restoration 512 or vice versa. Further, the difference 504 between the crowns may be a measure of a difference in features of the crowns, contact points, morphology, geometry and/or other characteristic features of the proposal compared to those of the omitted tooth.

In an aspect herein, a training 3D jaw model may include a plurality of healthy teeth. Thus, the training may be performed for a training 3D jaw model using the plurality of healthy teeth one after the other until all healthy teeth have been used as input for training the M/I model 310. Alternatively, a plurality of healthy teeth may be used as input at the same time depending on the structure of the m/l model 310 used. For example, this approach may be used to train a model to generate bridges.

More generally, data extraction module 402 of FIG. 4 may thus extract data from data store 416 and partition the data into training data 406 and validation data 410. One or more of the modified training 3D jaw models 418 may be used as at least part of the input 412 (training or validation input) and one or more omitted teeth 408 or healthy teeth 506 (such as from the pre-operative scan) may be used as targets 414 (training or validation targets). Training data may be used to train the model, while validation data may be used to tune the machine learning model's hyperparameters and make decisions about the model's structure, such as selecting between different architectures. Further, test data may be used to evaluate the final performance of the machine learning model and to estimate its generalization ability to new, unseen data. Thus, a supervised machine learning model of any architecture that is sufficiently data-hungry to require not just more training data but also more diverse teeth types and teeth characteristics training data than is practical and affordable to obtain using already designed restorations from dental practitioners can be developed and well maintained at exponentially more manageable, more effective and less time-consuming rates. Further, a set of artificially designed restorations without input from real teeth may differ significantly from real teeth morphologies and form at most only a subset of the variety of real tooth morphologies. Methods described herein may prevent or alleviate these concerns.

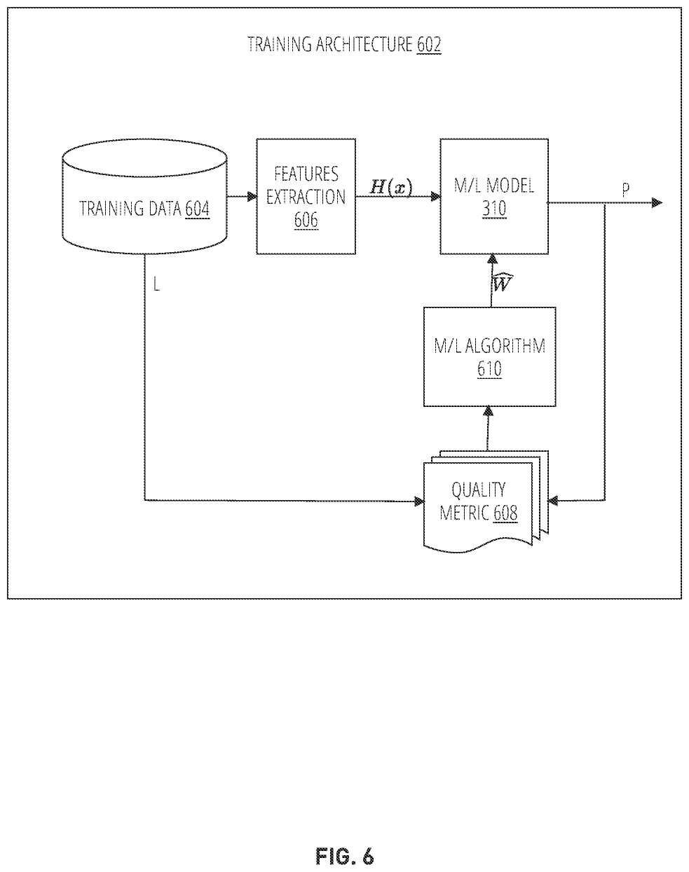

FIG. 6 shows an example training architecture 602. In an embodiment, upon receiving a request to provide a proposal, an application creates a set of values (such as an array) that are input to the input neurons of the m/l model 310 to produce a set of values that includes the output restoration 316. As shown in FIG. 6, which depicts a block diagram of an example training architecture 602 for machine-learning based proposal generation in accordance with an illustrative embodiment, program code extracts various features/attributes 606 from training data 604 with the training data entries having labels L. The features are utilized to develop a predictor function, H(x) or a hypothesis, which the program code utilizes as a m/l model 310. In identifying various features/attributes in the training data 604, the program code may utilize various techniques including, but not limited to, mutual information, which is an example of a method that can be utilized to identify features in an embodiment. Other embodiments may utilize varying techniques to select features, including but not limited to, principal component analysis, diffusion mapping, a Random Forest, and/or recursive feature elimination (a brute force approach to selecting features), to select the features. “P” is the output (e.g., restoration materials parameters and/or probabilities) that can be obtained, which when received, could further trigger the dental system to perform other steps such as display options or start a machining process. The program code may utilize a machine learning algorithm 610 to train m/l model 310, including providing weights for the outputs, so that the program code can prioritize various changes based on the predictor functions that comprise the m/l model 310.

The output can be evaluated by a quality metric 608.

By selecting a diverse set of training data 604, the program code trains m/l model 310 to identify and weight various attributes of different types of teeth. To utilize the m/I model 310, the program code obtains (or derives) input data or features to generate a set of values to input into input neurons of a neural network. Responsive to these inputs, the output neurons of the neural network produce a set that includes the output restoration 316 that may be presented contemporaneously on a display.

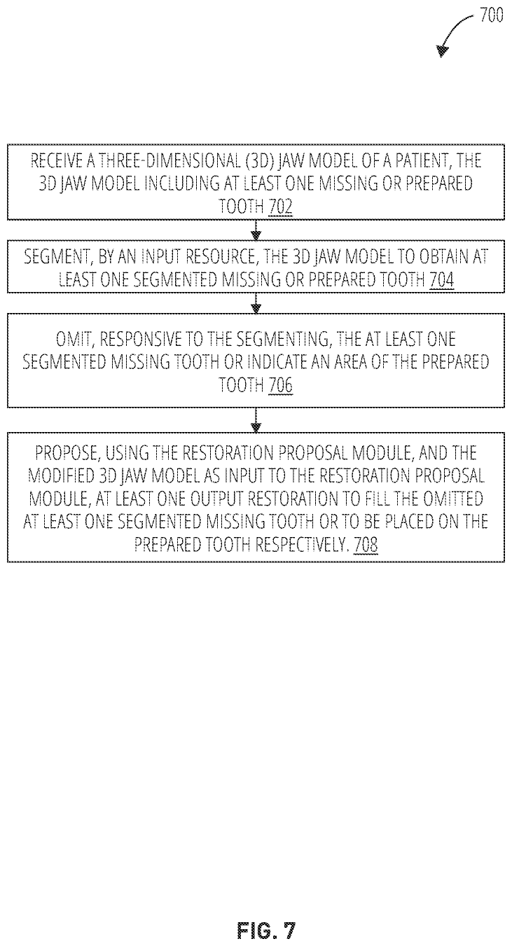

FIG. 7 discloses a flow chart illustrating a routine 700 in accordance with one or more embodiments. The routine may be performed by the machine learning engine 126.

In block 702, 126 may receive a three-dimensional (3D) jaw model of a of a patient, the 3D jaw model including at least one missing or unhealthy tooth. In block 704, machine learning engine 126 may segment, by an input resource, the 3D jaw model to obtain at least one segmented missing or prepared teeth. In block 706, machine learning engine 126 may omit, responsive to the segmenting, the at least one segmented missing teeth or may indicate an area of the prepared teeth from the 3D jaw model to obtain a modified 3D jaw model. Segmentation may be optional, in which case the model would be trained with large amounts of data sufficient enough to accurately predict restorations without information about locations of missing teeth or tooth preparations. In block 708, machine learning engine 126 may propose, using the restoration proposal module 320, with the modified 3D jaw model 328 as input to the restoration proposal module 320, at least one output restoration 316 to fill the omitted at least one segmented missing tooth or to be placed on the prepared teeth respectively.

In the routine, the at least one missing or prepared tooth may include at least one extracted tooth or at least one tooth that previously included a cavity, respectively. The at least one output restoration may be a surface representation (such as a triangle mesh, quad mesh, a point model or a heightfield) or a volume representation (such as a voxel or tetrahedral mesh). The modified 3D jaw model may also be a surface representation (such as a triangle mesh, a quad mesh, a point model or a heightfield) or a volume representation (such as a voxel or tetrahedral mesh). In some cases, the restoration proposal module 320 may further propose at least one tooth number corresponding to the at least one output restoration. In addition to obtaining the 3D jaw model from a storage database, the 3D jaw model may be obtained in real time by scanning the teeth of a patient who needs a restoration.

In another aspect of the routine, the restoration proposal module 320 may be trained using at least data from a continuously growing dataset of jaw situations and/or tooth morphologies. Other technical features may be readily apparent to one skilled in the art from the following figures, descriptions, and claims.

Thus, a computer implemented method, system or apparatus, and computer program product are provided in the illustrative embodiments for intelligent restoration proposals and other related features, functions, or operations. Where an embodiment or a portion thereof is described with respect to a type of device, the computer implemented method, system or apparatus, the computer program product, or a portion thereof, are adapted or configured for use with a suitable and comparable manifestation of that type of device.

Where an embodiment is described as implemented in an application, the delivery of the application in a Software as a Service (SaaS) model is contemplated within the scope of the illustrative embodiments. In a SaaS model, the capability of the application implementing an embodiment is provided to a user by executing the application in a cloud infrastructure. The user can access the application using a variety of client devices through a thin client interface such as a web browser, or other light-weight client-applications. The user does not manage or control the underlying cloud infrastructure including the network, servers, operating systems, or the storage of the cloud infrastructure. In some cases, the user may not even manage or control the capabilities of the SaaS application. In some other cases, the SaaS implementation of the application may permit a possible exception of limited user-specific application configuration settings.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, including but not limited to computer-readable storage devices as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method comprising:
receiving a three-dimensional (3D) jaw model of a patient, the 3D jaw model including at least one missing or prepared tooth;
segmenting, by an input resource, the 3D jaw model to obtain at least one segmented missing or prepared tooth area;
responsive to the 3D jaw model including at least one missing tooth, omitting, the at least one segmented missing tooth area from the 3D jaw model to obtain a modified 3D jaw model;
responsive to the 3D jaw model including at least one prepared tooth, indicating a position of the at least one prepared tooth area to obtain the modified 3D jaw model; and
proposing, using the restoration proposal module, and the modified 3D jaw model as input to the restoration proposal module, at least one output restoration for the at least one segmented missing or prepared tooth;
wherein the restoration proposal module is operated as a machine learning engine.

2. The method of claim 1, wherein one or more other input data are provided to the restoration proposal module comprising at least one of: an identification of tooth features, one or more tooth numbers identifying one or more teeth of the 3D jaw model, one or more color textures of the one or more teeth, one or more types of morphology of the one or more teeth.

3. The method of claim 1, wherein the at least one output restoration is a surface representation or a volume representation.

4. The method of claim 1, wherein the restoration proposal module further proposes at least one tooth number corresponding to the at least one output restoration.

5. The method of claim 1, wherein the 3D jaw model is obtained by scanning a patient's teeth.

6. The method of claim 1, wherein the restoration proposal module is an artificial neural network that is at least one of a convolutional neural network, and a Pointnet.

7. The method of claim 1, wherein the restoration proposal module is trained by:
receiving a plurality of training 3D jaw models that each include at least one healthy and/or prepared training tooth;
for each training 3D jaw model of the plurality of training 3D jaw models:
segmenting the training 3D jaw model to identify at least one healthy and/or prepared training segmented tooth area corresponding to the at least one healthy and/or prepared training tooth;
responsive to the training 3D jaw model including at least one healthy training segmented tooth, omitting at least one healthy training segmented tooth area from the training 3D jaw model to obtain a modified training 3D jaw model;
responsive to the training 3D jaw model including at least one prepared training tooth, indicating a position of the at least one prepared training segmented tooth area to obtain the modified training 3D jaw model; and
providing the modified training 3D jaw model as input to the restoration proposal module;
proposing, using the restoration proposal module, at least one corresponding training output restoration for the at least one healthy and/or prepared training segmented tooth area;
measuring a difference between the at least one healthy and/or prepared training segmented tooth and the at least one corresponding training output restoration; and
updating parameters of the restoration proposal module and repeating the proposing until the measured difference is minimized.

8. The method of claim 7, wherein the restoration proposal module is trained in an ongoing fashion using at least data from a continuously growing dataset of jaw situations and/or tooth morphologies.

9. The method of claim 7, wherein each training 3D jaw model is further preprocessed to obtain at least one other input for training the restoration proposal module by at least one of:
a) estimating a jaw line of the training 3D jaw model by computing a midline along a contour of training 3D jaw model;
b) extracting feature information from the training 3D jaw model;
c) categorizing each tooth of the training 3D jaw model as healthy, unhealthy or prepared tooth;
d) computing an orientation of the training 3D jaw model;
e) receiving a tooth number for one or more teeth of the training 3D jaw model; and
f) computing one or more colored surface texture of a tooth of the training 3D jaw model; and
g) identifying a tooth of the training 3D jaw model to be treated.

10. The method of claim 9, further comprising:
providing the preprocessed training 3D jaw model as an input to train the restoration proposal module.

11. The method of claim 7, further comprising:
training the restoration proposal module using a plurality of the at least one healthy and/or prepared training tooth one at a time until all healthy and/or prepared teeth in the training 3D jaw model have been used for training.

12. The method of claim 1, further comprising providing parameters of the at least one output restoration to an operator.

13. A non-transitory computer-readable storage medium, the computer-readable storage medium including instructions that when executed by a computer, cause the computer to carry out a method comprising:
receiving a three-dimensional (3D) jaw model of a patient, the 3D jaw model including at least one missing or prepared tooth;
segmenting, by an input resource, the 3D jaw model to obtain at least one segmented missing or prepared tooth area;
responsive to the 3D jaw model including at least one missing tooth, omitting, the at least one segmented missing tooth area from the 3D jaw model to obtain a modified 3D jaw model;
responsive to the 3D jaw model including at least one prepared tooth, indicating a position of the at least one prepared tooth area to obtain the modified 3D jaw model; and
proposing, using the restoration proposal module, and the modified 3D jaw model as input to the restoration proposal module, at least one output restoration for the at least one segmented missing or prepared tooth;
wherein the restoration proposal module is operated as a machine learning engine.

14. The non-transitory computer-readable storage medium of claim 13, wherein the computer further carries out the method comprising:
training the restoration proposal module by:
receiving a plurality of training 3D jaw models that each include at least one healthy and/or prepared training tooth;
for each training 3D jaw model of the plurality of training 3D jaw models:
segmenting the training 3D jaw model to identify at least one healthy and/or prepared training segmented tooth area corresponding to the at least one healthy and/or prepared training tooth;
responsive to the training 3D jaw model including at least one healthy training segmented tooth, omitting at least one healthy training segmented tooth area from the training 3D jaw model to obtain a modified training 3D jaw model;
responsive to the training 3D jaw model including at least one prepared training tooth, indicating a position of the at least one prepared training segmented tooth area to obtain the modified training 3D jaw model; and
providing the modified training 3D jaw model as input to the restoration proposal module;
proposing, using the restoration proposal module, at least one corresponding training output restoration for the at least one healthy and/or prepared training segmented tooth area;
measuring a difference between the at least one healthy and/or prepared training segmented tooth and the at least one corresponding training output restoration; and
updating parameters of the restoration proposal module and repeating the proposing until the measured difference is minimized.

15. A computing system comprising:
a processor; and
a memory storing instructions that, when executed by the processor, configure the system to:
receive a three-dimensional (3D) jaw model of a patient, the 3D jaw model including at least one missing or prepared tooth;
segment, by an input resource, the 3D jaw model to obtain at least one segmented missing or prepared tooth area;
responsive to the 3D jaw model including at least one missing tooth, omit, the at least one segmented missing tooth area from the 3D jaw model to obtain a modified 3D jaw model;
responsive to the 3D jaw model including at least one prepared tooth, indicate a position of the at least one prepared tooth area to obtain the modified 3D jaw model; and
propose, using the restoration proposal module, and the modified 3D jaw model as input to the restoration proposal module, at least one output restoration for the at least one segmented missing or prepared tooth;
wherein the restoration proposal module is operated as a machine learning engine.

16. The computing system of claim 15, wherein the processor further:
trains the restoration proposal module by:
receiving a plurality of training 3D jaw models that each include at least one healthy and/or prepared training tooth;
for each training 3D jaw model of the plurality of training 3D jaw models:
segmenting the training 3D jaw model to identify at least one healthy and/or prepared training segmented tooth area corresponding to the at least one healthy and/or prepared training tooth;
responsive to the training 3D jaw model including at least one healthy training segmented tooth, omitting at least one healthy training segmented tooth area from the training 3D jaw model to obtain a modified training 3D jaw model;
responsive to the training 3D jaw model including at least one prepared training tooth, indicating a position of the at least one prepared training segmented tooth area to obtain the modified training 3D jaw model; and
providing the modified training 3D jaw model as input to the restoration proposal module;
proposing, using the restoration proposal module, at least one corresponding training output restoration for the at least one healthy and/or prepared training segmented tooth area;
measuring a difference between the at least one healthy and/or prepared training segmented tooth and the at least one corresponding training output restoration; and
updating parameters of the restoration proposal module and repeating the proposing until the measured distance is minimized.

17. The computing system of claim 16, wherein the processor trains the restoration proposal module in an ongoing fashion using at least data from a continuously growing dataset of jaw situations and/or tooth morphologies.

18. The computing system of claim 15, wherein the processor provides one or more other input data to the restoration proposal module comprising at least one of: an identification of tooth features, one or more tooth numbers identifying one or more teeth of the 3D jaw model, one or more colors of the one or more teeth, one or more types of morphology of the one or more teeth.

19. A method comprising:
receiving a three-dimensional (3D) jaw model of a patient, the 3D jaw model including at least one missing or prepared tooth;
proposing, using a restoration proposal module, and the 3D jaw model as input to the restoration proposal module, at least one output restoration for the at least one missing or prepared tooth;
wherein the restoration proposal module is operated as a machine learning engine.

20. The method of claim 19, wherein the restoration proposal module is trained by:
receiving a plurality of training 3D jaw models that each include at least one healthy and/or prepared training tooth;
for each training 3D jaw model of the plurality of training 3D jaw models:
proposing, using the restoration proposal module, at least one corresponding training output restoration for the at least one healthy and/or prepared training tooth;
measuring a difference between the at least one healthy and/or prepared training tooth and the at least one corresponding training output restoration; and
updating parameters of the restoration proposal module and repeating the proposing until the measured difference is minimized.
